(19) United States
(12) Patent Application Publication
(10) Pub. No.: US 2018/0161031 A1
(43) Pub. Date: Jun. 14, 2018

(54) SUTURE PACKAGING DEVICE

(71) Applicant: MERIL ENDO SURGERY PVT. LTD., Vapi (IN)

(72) Inventors: HEMCHANDRA M. SONAWANE, Chala (IN); PREMNATH S. GORNALE, Aurangabad (IN); RAJNIKANT GANDAL VYAS, Mumbai (IN); PRAMOD KUMAR MINOCHA, Vapi (IN)

(73) Assignee: MERIL ENDO SURGERY PVT. LTD., Vapi (IN)

(21) Appl. No.: 15/688,862

(22) Filed: Aug. 28, 2017

(30) Foreign Application Priority Data
Dec. 14, 2016 (IN) 201621042670

(51) Int. Cl. A61B 17/06

(52) U.S. Cl. CPC A61B 17/06114; A61B 2017/06157; A61B 2017/06142; A61B 17/06004

(57) ABSTRACT

A suture tray having an upper component with a top surface and a bottom surface is disclosed. The tray also includes a plurality of flexible members integrally coupled to the upper component such that the flexible members have a clearance for assisting easy lift-press mechanism and a lower component having an outer peripheral wall and inner peripheral wall. The outer and inner peripheral wall mate with the upper component to form a winding channel. The tray also includes a secondary female locking member integrally coupled to the inner peripheral wall, the secondary female locking member mates with a secondary male locking member integrally formed on the upper component to additionally lock the assembly.

### FIELD OF INVENTION

The present disclosure relates to a packaging device. More particularly, the present disclosure relates to a packaging device for surgical sutures.

### BACKGROUND

Conventional suture packages include multiple functionalities like protecting a needle and suture assembly during operation, storage and transportation, dispensing of sutures during surgery or other medical procedures, etc. Typically, a suture package may be armed with a surgical needle. Alternatively, the suture package may include a surgical suture without any needle. The suture package facilitates effective packaging of sutures, which may avoid any tangled structure in the suture.

Of late, suture packages may include a two piece structure having a winding channel. The two piece structure includes an upper component and a lower component. The periphery of the upper component has a plurality of flexible projections that mate with the lower component to form a winding channel. The flexible projections are spaced apart at minimal intervals. Such packages are typically molded from plastic, and are mounted on a winding fixture with the sutures, wherein the winding fixture holds the suture package and assists a wounding device for wounding the suture in the winding channel of the suture package.

While winding the suture in the package, the wounding device lifts the closely provisioned flexible projections and packs the suture in the winding channel of the suture package, and then closes the flexible projections to seal the suture. As the wounding device lifts and closes the flexible projections at a high speed, the closely packed structure of the flexible projections may tend to break due to space constraints and friction.

### SUMMARY

The main aspect of the invention relates to a suture tray. The suture tray includes an upper component having a top surface and a bottom surface; a plurality of flexible members integrally coupled to the upper component, the flexible members having a clearance for assisting easy lift-press mechanism; a lower component having an outer peripheral wall and inner peripheral wall, the outer and inner peripheral wall mates with the upper component to form a winding channel; and a secondary female locking member integrally coupled to the inner peripheral wall, the secondary locking member mates with a secondary male locking member integrally formed on the upper component to additionally lock the assembly.

The other aspect of the invention relates to an arcuate structure of the flexible member. The arcuate structure facilitates effective gripping of the suture in the suture package. Further, the arcuate structure provides easy access for the suture winding machine, which lifts and presses the arcuate flexible member while wounding the suture. In an embodiment, the arcuate structure of the flexible member may vary in the range of 0.58 mm to 0.62 mm.

Yet another aspect of the invention relates to a variable angle and distance between the flexible members. The flexible members are positioned at an angle with respect to each other. In one embodiment, the flexible members may be at an angle between 0 to 45 degrees with respect to the corresponding radius (shown in FIG. 1a) and with a clearance 305 (depicted in FIG. 3a) in the range of 1 to 3 mm. The variably angled profile of the flexible members facilitates an effective gripping force to the suture.

Yet another aspect of the invention relates to a curved profile of the suture tray. The curved profile of the tray is in-phase with the travel path of the suture, and provides a smooth wounding of the suture. Also, during usage of the suture package, the curved profile of the tray enables the suture to be pulled from the suture package at ease and without any bend. In one embodiment, the curved profile of the tray is in the range of Inner wall min Radius 9 mm and maximum Radius 197 mm and outer wall min Radius 16 mm and maximum radius 206 mm.

Yet another aspect of the invention relates to a flat tunnel profile of the suture tray. The flat tunnel profile of the suture tray facilitates the suture to travel in the suture tray without any tangling; and released from the suture as a straight suture without any bend.

### BRIEF DESCRIPTION OF DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the disclosure is not limited to specific devices, methods and instrumentalities disclosed herein. Moreover, those in the art will understand that the drawings are not to scale. Wherever possible, like elements have been indicated by identical numbers.

FIGS. 1a-1b illustrate an exemplary suture tray device for packaging a suture and a needle, in accordance with an embodiment of the present disclosure;

FIG. 2 illustrates a top view of a upper component of the suture tray, in accordance with an embodiment of the present disclosure;

FIG. 3A illustrates a segmental view of a flexible projection in the upper component of the suture tray, in accordance with an embodiment of the present disclosure;

FIG. 3B illustrates a zoomed view of a tip of a flexible projection of FIG. 3A, in accordance with an embodiment of the present disclosure;

FIG. 3C illustrates a base of the upper component of the suture tray, in accordance with an embodiment of the present disclosure;

FIG. 3d illustrates a segmental isometric view of a male connector in the upper component of the suture tray, in accordance with a first embodiment of the present disclosure;

FIG. 3e illustrates a segmental isometric view of a pillar in the upper component of the suture tray, in accordance with a first embodiment of the present disclosure;

FIG. 3f illustrates a segmental isometric view of a needle locking member in the upper component of the suture tray, in accordance with a first embodiment of the present disclosure;

FIG. 4 illustrates a top view of a lower component of the suture tray, in accordance with an embodiment of the present disclosure;

FIG. 5a illustrates a segmental isometric view of a boundary element in the lower component of the suture tray, in accordance with an embodiment of the present disclosure;

FIG. 5b illustrates a segmental isometric view of a first protrusion element in the lower component of the suture tray, in accordance with an embodiment of the present disclosure;

FIG. 5c illustrates a segmental isometric view of a winding channel base 103 in the lower component of the suture tray, in accordance with an embodiment of the present disclosure;

FIG. 5d illustrates a segmental isometric view of a second protrusion element in the lower component of the suture tray, in accordance with an embodiment of the present disclosure; and

FIG. 5e illustrates a segmental isometric view of a female connector in the lower component of the suture tray, in accordance with an embodiment of the present disclosure.

### DETAILED DESCRIPTION

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; Definitions of certain words and phrases are provided throughout this patent document, and those of ordinary skill in the art will understand that such definitions apply in many, if not most, instances to prior as well as future uses of such defined words and phrases.

Wherever possible, same reference numbers will be used throughout the drawings to refer to same or like parts. Moreover, references to various elements described herein are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

Particular embodiments of the present disclosure are described herein below with reference to the accompanying drawings, however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

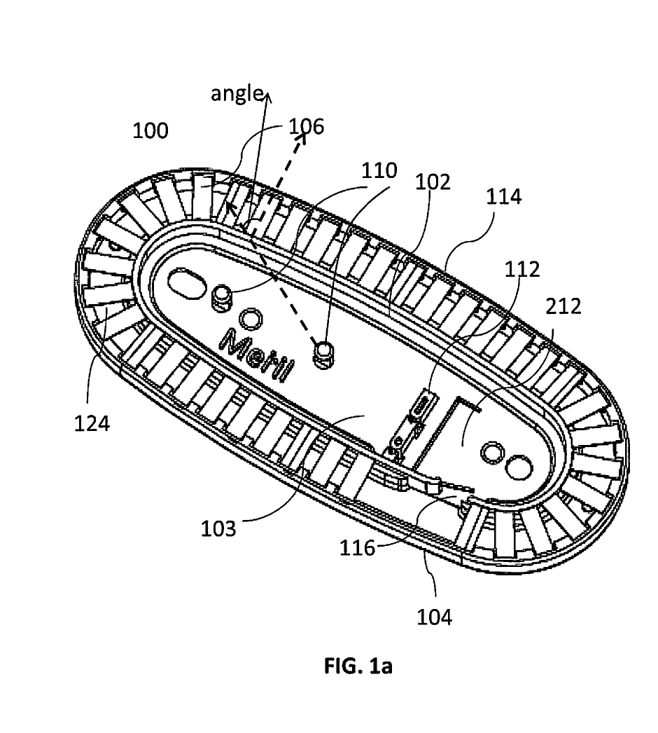

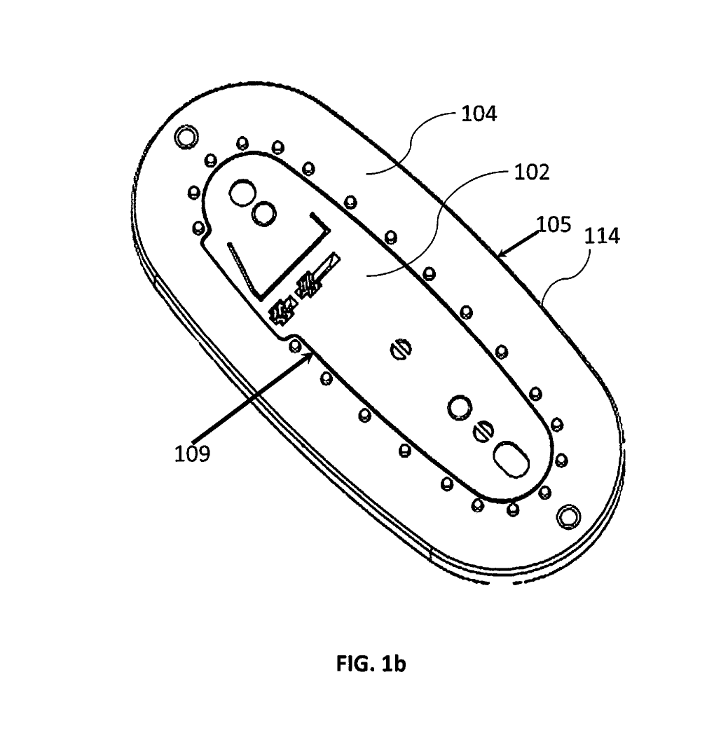

FIG. 1a shows a perspective view of an exemplary suture tray 100. The suture tray includes an upper component 102 coupled to a lower component 104. The upper component 102 is formed of two sections—a base 103 and a plurality of flexible projections 106. The base 103 includes multiple pillars 110, a needle locking member 112 and a flap 212. The plurality of flexible projections 106 are attached to the periphery of the base 103. The upper component 102 is assembled with the lower component 104 to form a winding channel 124. A winding device (not shown) packs a suture (not shown) in the suture tray 100 through the winding channel 124. The ‘flexible projection’ and the ‘flexible member’ refer to same structure and are functionally equivalent.

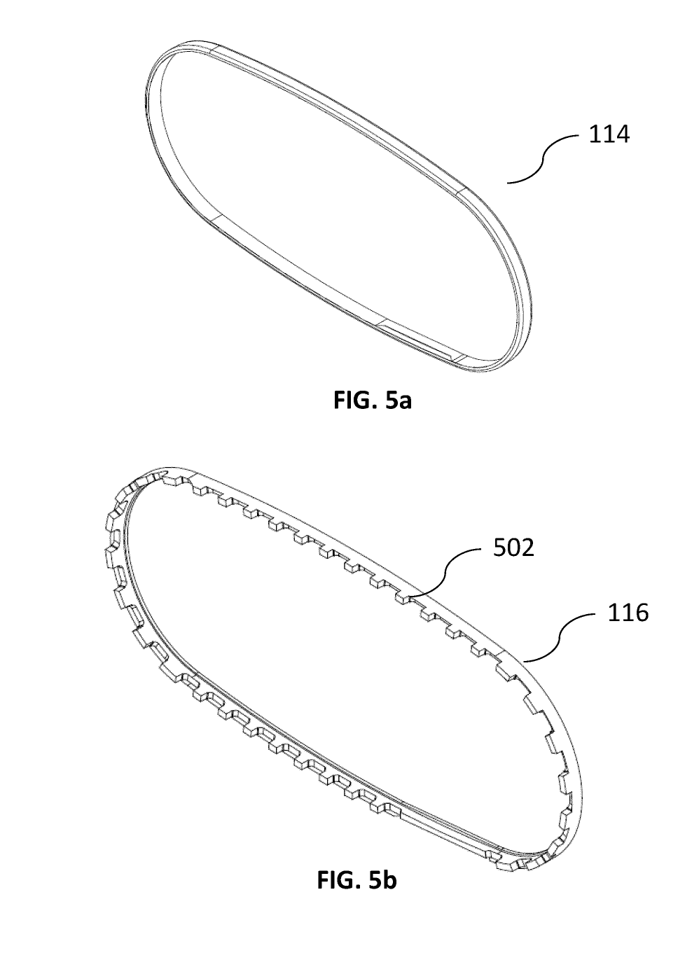

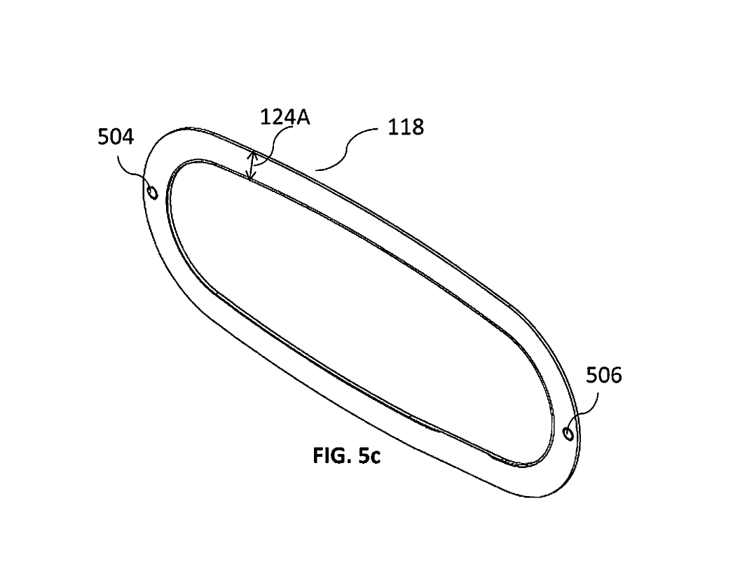

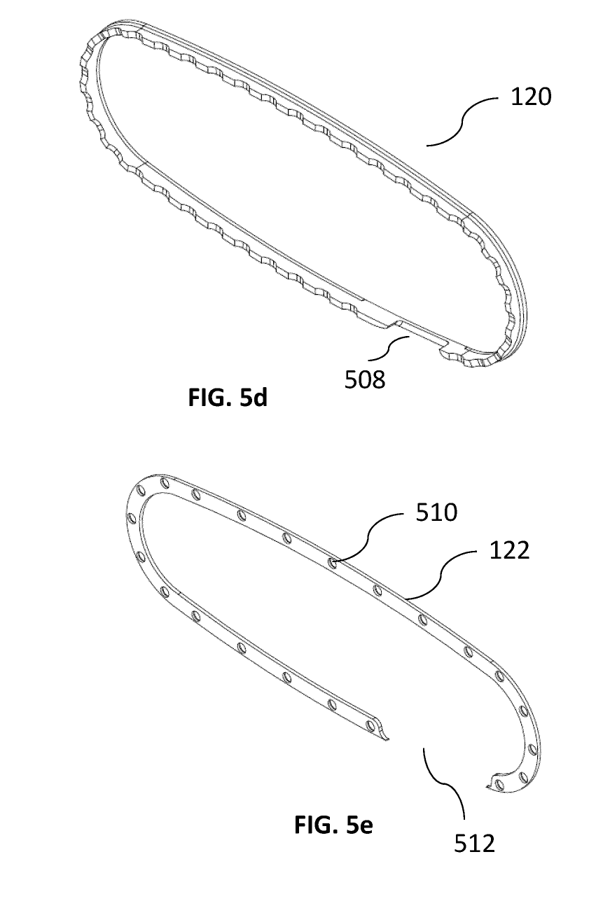

The winding channel 124 has a flat tunnel profile 124A (depicted in FIG. 5c) in the suture tray 100. The flat tunnel profile 124A (depicted in FIG. 5c) of the winding channel 124 facilitates the suture to travel in the suture tray without any tangling; and released from the suture tray as a straight suture without any bend(s).

FIG. 1b shows a bottom view of the exemplary suture tray 100. The bottom view illustrates the lower component 104 mounted on the upper component 102. The lower component 104 includes a boundary element 114. The lower component 104 has an outer peripheral wall 105 and inner peripheral wall 109. The outer and inner peripheral wall 105, 109 respectively mate with the upper component 102 to form the winding channel 124 which holds a suture. This assembly acts as a cage to protect the suture, which is wounded in a winding channel via a winding device explained below in detail.

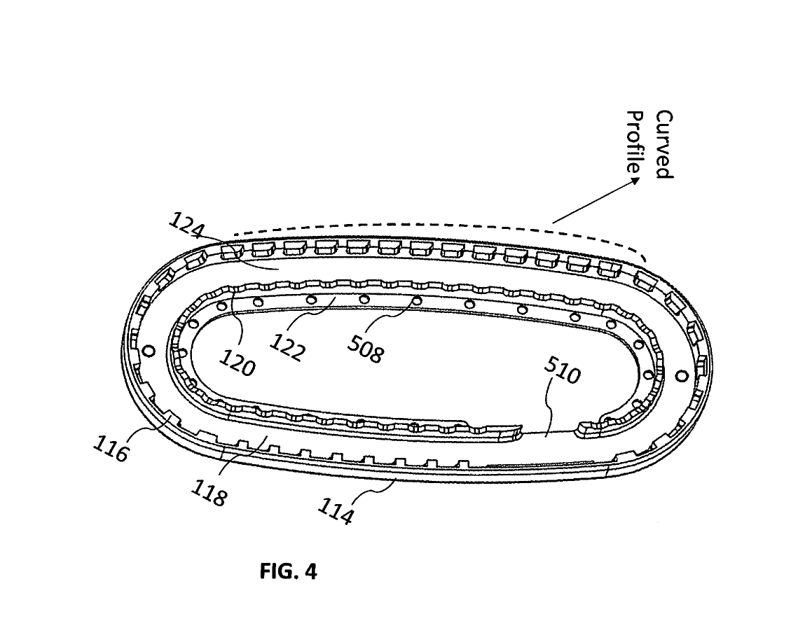

The lower component 104 further includes, without limitation, a first protrusion element 116, a winding channel base 118, a second protrusion element 120 and a female connector 122 as explained in FIG. 4.

The lower component 104 has a curved profile, for example, an oval profile. The curved profile of the lower component 104 is in-phase with the travel path of the suture, and provides a smooth wounding of the suture. Also, during usage of the suture package, the curved profile of the lower component 104 enables the suture to be pulled from the suture package at ease and without one or more bends. In one embodiment, radius of inner peripheral wall 109 of the tray ranges from 9 mm to 197 mm while radius of outer peripheral wall 105 (depicted in FIG. 1b) ranges from 16 mm to 206 mm.

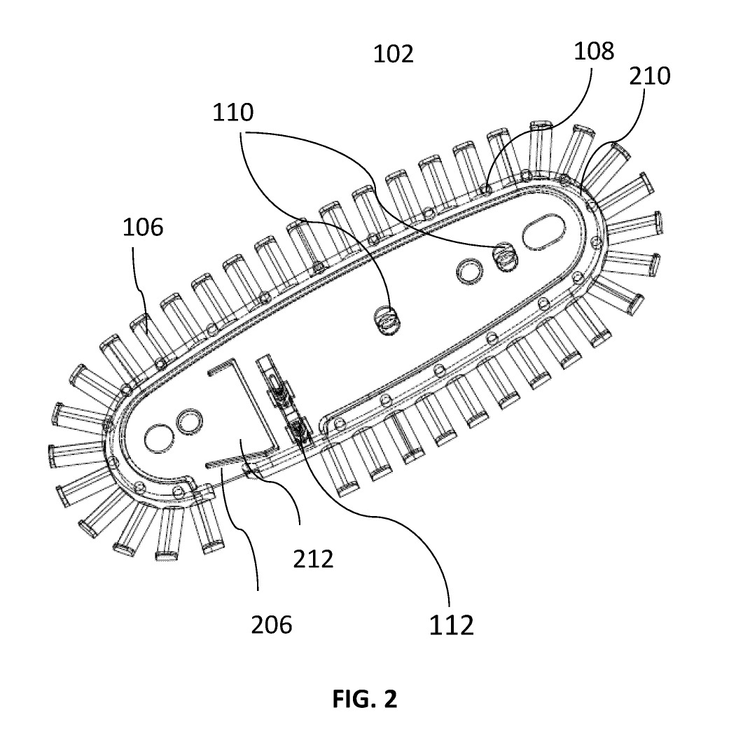

FIG. 2 illustrates a detailed two-dimensional diagram of the upper component 102 of the suture tray 100 in accordance with an embodiment of the present disclosure. The upper component 102 has a top surface and a bottom surface. FIG. 2 depicts the bottom surface or base 103 of the upper component.

The base 103 includes multiple male connectors 108 at its boundary 210. Further, the male connector 108 is adjacently aligned with respective flexible projection 106. In an embodiment, the male connectors 108 are protrusions that extend away from the surface of the base 103.

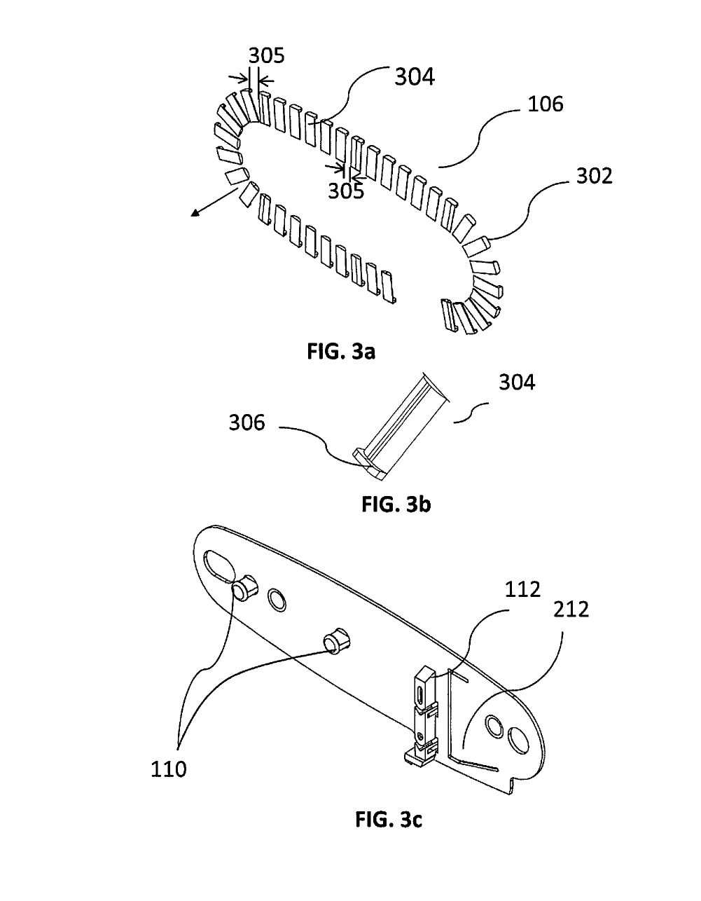

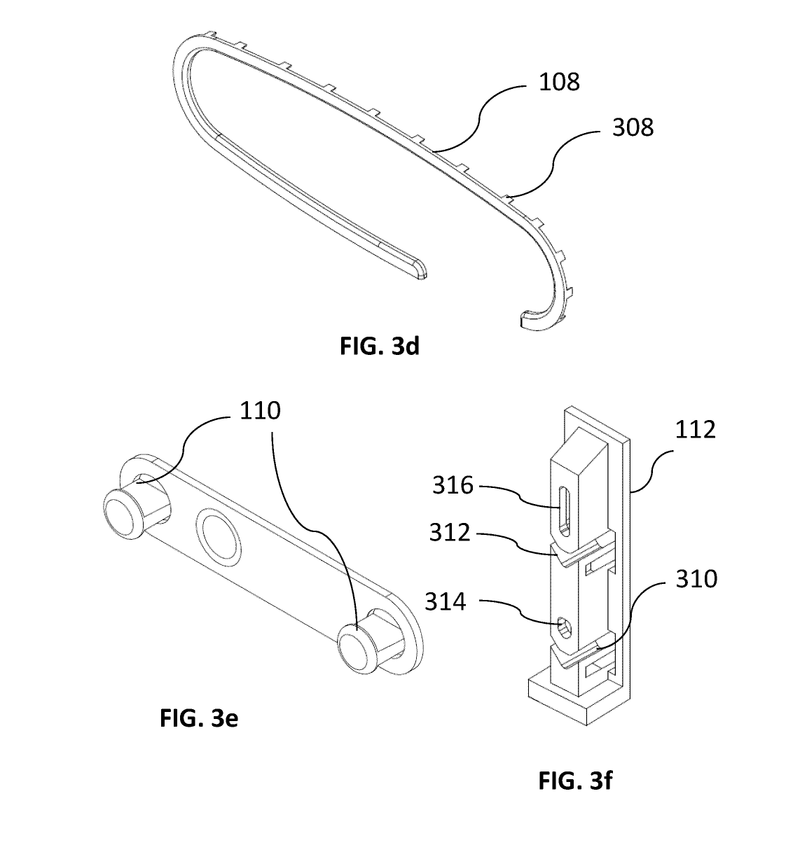

The base 103 has one or more pillars 110, a flap 212, and the needle locking member 112. The pillars 110 and the needle locking member 112 having a finite height and are projected away from the surface of the base 103 of the upper component 102. The needle locking member 112 is further described in FIG. 3f.

The flap 212 is located proximate the needle locking member 112, such that, the flap 212 serves to lift the needle from the tray.

FIGS. 3a-3f depict different elements of the upper component in accordance with an embodiment of the present disclosure.

FIG. 3a depicts the flexible projections 106 attached to the periphery of the base 103 of the upper component 102 of the suture tray 100. Each flexible projection 106 includes a tip 302 and a leave 304. The tip 302 is engaged to the respective first protrusion element 116 of the lower component 104. Unlike conventional flexible projections of suture packaging devices, there is adequate clearance 305 (depicted in FIG. 3a) between any two adjacent leaves 304 of the respective flexible projections 106. Adequate clearance may be in the range of 1 mm to 3 mm.

The flexible projections 106 are positioned at an angle with respect to each other. For example, the flexible projections 106 may be at an angle between 0 to 45 degrees with respect to the corresponding radius (shown in FIG. 1a) and with a clearance 305 (depicted in FIG. 3a) in the range of 1 to 3 mm. The variably angled profile of the flexible members facilitates an effective gripping force to the suture.

In an embodiment, one or more leaves 304 of the flexible projections 106 have an arcuate structure (FIG. 3b). The arcuate structure 306 (FIG. 3b) of the leaves 304 facilitates effective coupling between the upper component 102 and the bottom component 104. The arcuate structure 306 (FIG. 3b) of the leaves 304 of flexible projections 106 assists in easy lift-press mechanism that avoids any breakage of leaves from the suture tray 100 during a winding process. In one embodiment, the radius of curvature of the arcuate structure may vary from 0.58 mm to 0.62 mm. However, the leaves 302 may have any other structure such as, trapezoidal.

The arcuate structure 306 facilitates effective gripping of the suture in the suture package 100. Further, the arcuate structure 306 provides easy access for the suture winding machine, which lifts and presses the arcuate leaves 304 while wounding the suture. In an embodiment, the arcuate structure 306 of the leaves 304 may vary in the range of 0.58 mm to 0.62 mm.

FIG. 3c depicts the base of the upper component 102. The base 103 forms the central portion of the upper component to which flexible projections 106 are attached. Specifically, the projections 106 are attached to the periphery of the base 103. In an embodiment, the base 103 is oval in structure. The base 103 is made of HDPE/PP. The base 103 acts as a support for the needle locking member 112 and the pillars 110. In an embodiment, a portion of the base 103 does not follow the oval contour rather it is extended to support a paper lid which contains for example product information.

FIG. 3d illustrates the male connector 108 strip of the upper component 102. In an embodiment, the male connector 108 has a plurality of projections 308. Such projections 308 of the male connector 108 are coupled to the corresponding female connectors 122 (FIG. 5e) in the lower component 104. The material of the male connector is HDPE/PP.

FIG. 3e depicts the pillar 110 provided on the base 103. In an embodiment, as depicted, two pillars are provided. However, based upon requirement the number of pillars may vary. The pillars 110 provided on the top surface 202 of the upper component 102 have a rectangular pin shaped structure which facilitates the clamping of a label (not shown) on the upper component 102. The label may include a bibliographic data of the tray 100. The bibliographic data may include, but not limited to, a cost, a dimension, or a usage instruction of the tray 100.

FIG. 3f depicts a needle locking member. The needle locking member 112 has a rectangular shape with a first rest location 310 and a second rest location 312 for resting a needle (not shown). The first rest location 310 and the second rest location 312 have a V-shaped structure and assist in holding the needle as shown in FIG. 2. A hole 314 is provided on top of the first rest location 310 and a slot 316 is provided on top of the second rest location 312.

Referring to FIG. 4, it illustrates the lower component 104 of the suture tray 100. The periphery of the lower component 104 of the suture tray includes the boundary element 114. The boundary element 114 (depicted in FIG. 5a) defines a wall of the lower component 104. The lower component 104 includes without limitation a first protrusion element 116, a winding channel base 118, and a winding channel 124 each of which are explained in detail in subsequent diagrams. The first protrusion element 116 is integrally coupled to the boundary element 114.

FIG. 5b depicts an exemplary first protrusion element 116. The first protrusion element 116 has a plurality of protrusions 502 that engage with the tip 302 of the corresponding leaves 304 of the flexible projections 106 of the upper component 102 to form the winding channel 124. The first protrusion element 116 does not have any protrusion proximate to the suture guide 206.

FIG. 5c shows the winding channel base 118 which forms the base for the winding channel 124. The winding channel base 118 includes a first hole 504 at a proximal end and a second hole 506 at a distal end to mate with a first protrusion (not shown) and a second protrusion (not shown) of the upper component 102. A first end of the winding channel base 118 is integrally coupled to the boundary element 114. Further, a second end of the winding channel base 118 integrally couples the second protrusion element 120. The second protrusion element 120 has a slot 508 proximate the suture guide 206 for guiding the suture out of the suture tray 100 as shown in FIG. 5d.

FIG. 5e shows the female connector 122 that mates with the corresponding male connector 108 to form additional locking between the upper component 102 and the lower component 104. The female connector 122 includes a plurality of holes 510 which mate with the plurality of protrusions 308 of the male connector 108 to form a lock assembly that additionally secures the winding channel 124 of the suture tray 100. The female connector 122 has a slot 512 proximate the suture guide 206 for guiding the suture out of the suture tray 100.

The suture tray 100 is preferably in oval structure. However, the suture tray 100 may adopt any other structure other than the oval structure, such as, a circular, a polygonal, a rectangular or a profile with filleted/chamfered corners and the like. The suture tray 100 is made of plastic. In one embodiment, the suture tray 100 may be composed of HDPE/PP.

In an exemplary illustration of the functioning of the device 100, during lift-press mechanism, first flexible projections 106 are lifted using a winding device, and thereafter a suture is inserted in the winding channel 124. Then upon inserting the suture in the winding channel 124, the winding device presses the flexible projections 106 that are lifted during the insertion of the suture. As the design of the flexible projections 106 has adequate clearance 305, during lifting and dropping the flexible projections 106, the breakage or excessive friction of the flexible projections 106 during winding process is negligible.

It will be apparent from the foregoing that, while particular forms of the invention have been illustrated and described, various modifications can be made without departing from the spirit and scope of the invention. Accordingly, it is not intended that the invention be limited, except as by the appended claims.

1. A suture tray, comprising:
a upper component having a top surface and a bottom surface;
a plurality of flexible members integrally coupled to the upper component, the flexible members having a clearance for assisting a lift-press mechanism, wherein the clearance is in a range of 1 to 3 mm;
a lower component having an outer peripheral wall and inner peripheral wall, the outer and inner peripheral walls mate with the upper component to form a winding channel; and
a female connector integrally coupled to the inner peripheral wall, the female connector mates with a male connector integrally formed on the upper component to lock the upper component and the lower component.

2. The suture tray as claimed in claim 1, wherein the flexible members have an arcuate structure configured to hold the suture.

3. The suture tray as claimed in claim 2, wherein the arcuate structure of the flexible members may vary in a range of 0.58 mm to 0.62 mm.

4. (canceled)

5. The suture tray as claimed in claim 1, wherein the clearance between any two consecutive flexible members of the plurality of flexible members is in the range of 1 mm to 3 mm to provide gripping of the suture.

6. The suture tray as claimed in claim 1, wherein each flexible member of the plurality of flexible members is radially angled with respect to a corresponding radius in a range of 0 to 45 degrees.

7. The suture tray as claimed in claim 1, wherein the winding channel has a flat base for holding a suture without any bend.

8. The suture tray as claimed in claim 1, wherein the lower component has a curved profile, which assists of the suture tray.

9. The suture tray as claimed in claim 1, wherein the lower component has a curved profile, which assists delivery of suture from the suture tray.
